(19) United States
(12) Patent Application Publication
(10) Pub. No.: US 2018/0125405 A1
(43) Pub. Date: May 10, 2018

(54) MENTAL STATE ESTIMATION USING FEATURE OF EYE MOVEMENT

(71) Applicant: INTERNATIONAL BUSINESS MACHINES CORPORATION, Armonk, NY (US)

(72) Inventor: Yasunori Yamada, Saitama (JP)

(21) Appl. No.: 15/345,845

(22) Filed: Nov. 8, 2016

(51) Int. Cl. A61B 5/16; A61B 3/113; A61B 5/00; A61B 5/11; A61B 3/11; A61B 3/00; A61B 5/18

(52) U.S. Cl. CPC A61B 5/165; A61B 3/113; A61B 5/7267; A61B 5/18; A61B 3/112; A61B 3/0091; A61B 5/1103

(57) ABSTRACT

A computer-implemented method for estimating a mental state of a target individual includes obtaining information of eye movement of the target individual in a coordinate system, in which the coordinate system determines a point representing eye movement by an angle and/or a distance with respect to a reference point that is related to a center of an object showing a scene, analyzing the information of the eye movement to extract a feature of the eye movement defined in relation to the coordinate system, and estimating the mental state of the target individual using the feature of the eye movement.

### BACKGROUND

### Technical Field

The present invention, generally, relates to mental state estimation, and more particularly to techniques for estimating a mental state of an individual and training a learning model that is used for estimating a mental state of an individual.

### Related Art

Mental fatigue is of increasing importance to improve health outcomes and to support the aging population. The costs of fatigue-related accidents and errors are estimated to be a considerable amount in society. Mental fatigue is also an important symptom in general practice due to its association with a large number of chronic medical conditions. Hence, there is a need for techniques for estimating a mental state such as mental fatigue to obviate a risk of accidents and errors and/or to early detection of disease.

Eye movement features acquired during a task, such as driving, have been used to develop mental state estimation systems. However, there are a few examples that can be applicable to natural viewing conditions where a subject watches a video clip while not performing any cognitive task. Also accuracy of mental state estimation is desired to be improved.

### SUMMARY

According to an embodiment of the present invention, a computer-implemented method for estimating a mental state of a target individual is provided. The method includes obtaining information of eye movement of the target individual in a coordinate system, in which the coordinate system determines a point representing the eye movement by an angle and/or a distance with respect to a reference point that is related to a center of an object showing a scene. The method also includes analyzing the information of the eye movement to extract a feature of the eye movement defined in relation to the coordinate system. The method further includes estimating the mental state of the target individual using the feature of the eye movement.

According to another embodiment of the present invention, a computer-implemented method for training a learning model that is used for estimating a mental state of a target individual is provided. The method includes preparing information of eye movement of a participant in a coordinate system, in which the coordinate system determinines a point representing the eye movement by an angle and/or a distance with respect to a reference point that is related to a center of an object showing a scene. The method also includes extracting a feature of the eye movement defined in relation to the coordinate system by analyzing the information of the eye movement. The method further includes training the learning model using one or more training data, each of which includes the feature of the eye movement and corresponding label information that indicates mental state of the participant.

Computer systems and computer program products relating to one or more aspects of the present invention are also described and claimed herein.

Additional features and advantages are realized through the techniques of the present invention. Other embodiments and aspects of the invention are described in detail herein and are considered a part of the claimed invention.

### BRIEF DESCRIPTION OF THE DRAWINGS

The subject matter, which is regarded as the invention, is particularly pointed out and distinctly claimed in the claims at the conclusion of the specification. The forgoing and other features and advantages of the invention are apparent from the following detailed description taken in conjunction with the accompanying drawings in which:

FIG. 1 illustrates a block/flow diagram of a mental fatigue estimation system according to an exemplary embodiment of the present invention;

FIG. 2A depicts an example of a mental fatigue estimation model according to an embodiment of the present invention;

FIG. 2B depicts an example of a mental fatigue estimation model according to an embodiment of the present invention;

FIG. 2C depicts an example of a mental fatigue estimation model according to an embodiment of the present invention;

FIG. 3A illustrates an example of a coordinate system used for extracting one or more extended features according to an embodiment of the present invention;

FIG. 3B depicts an example of one or more extended features defined in relation to the coordinate system according to an embodiment of the present invention;

FIG. 4 is a flowchart depicting a process for learning a mental fatigue estimation model according to an embodiment of the present invention;

FIG. 5 is a flowchart depicting a process for estimating mental fatigue using the trained mental fatigue estimation model according to an embodiment of the present invention;

FIG. 6 is a flowchart depicting a process for estimating mental fatigue according to an embodiment of the present invention; and

FIG. 7 depicts a computer system according to an embodiment of the present invention.

### DETAILED DESCRIPTION

The present invention will be described using particular embodiments, and the embodiments described hereafter are understood to be only referred as examples and are not intended to limit the scope of the present invention.

One or more embodiments according to the present invention are directed to computer-implemented methods, computer systems and computer program products for estimating a mental state of a target individual using a feature of eye movement obtained from a target individual. One or more other embodiments according to the present invention are directed to computer-implemented methods, computer systems and computer program products for training a learning model using a feature of eye movement obtained from a participant, in which the learning model can be used for estimating a mental state of a target individual.

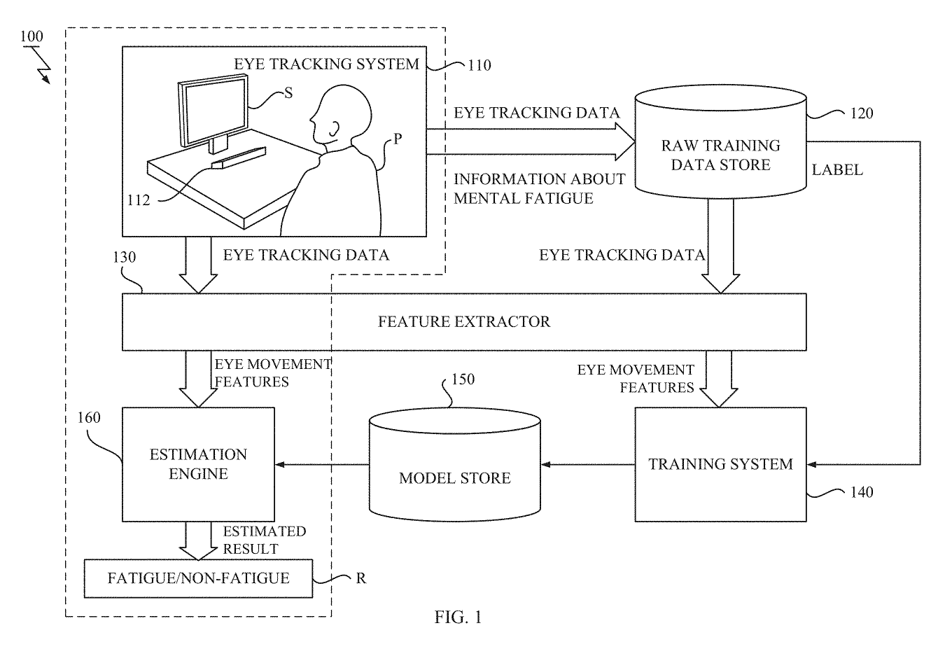

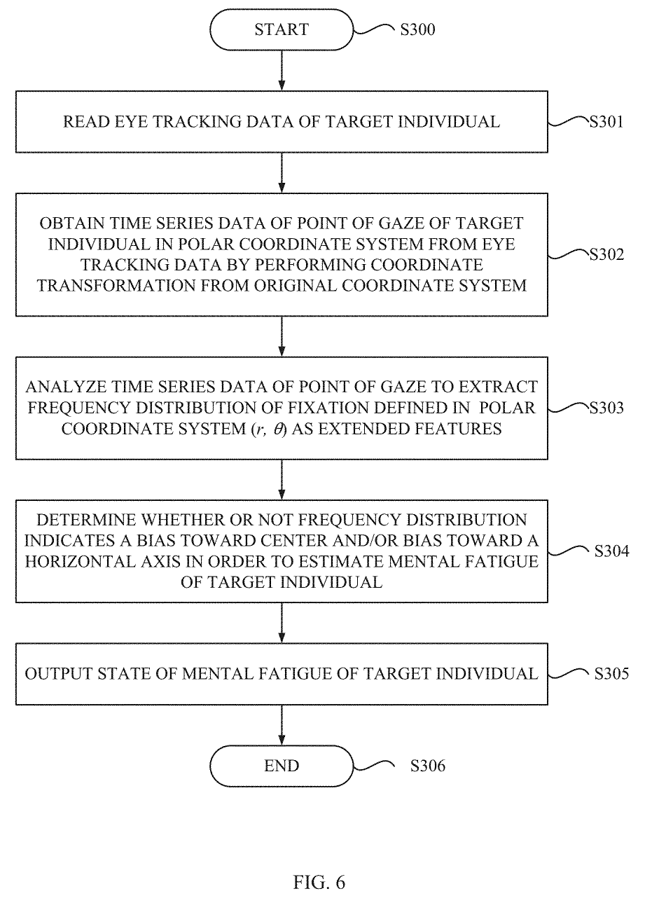

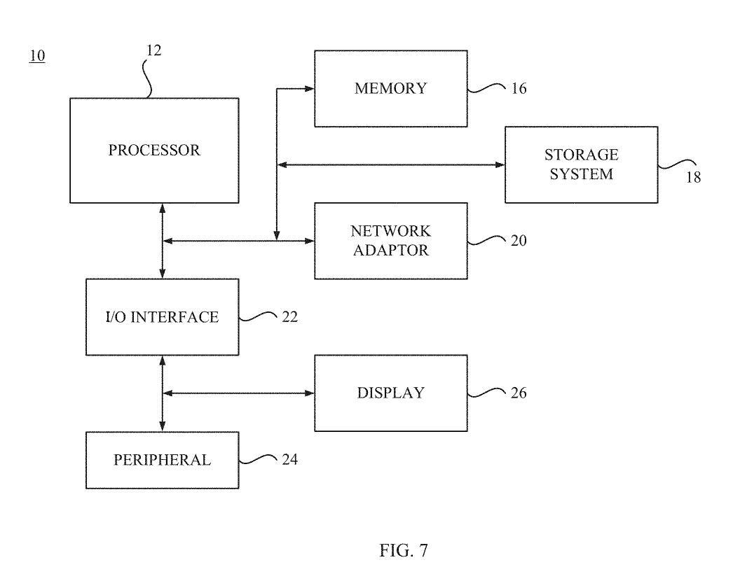

Hereinafter, referring to the series of FIGS. 1-5, a computer system and methods for training a mental fatigue estimation model and estimating mental fatigue of a target individual by using the mental fatigue estimation model according to an exemplary embodiment of the present invention will be described. Then, referring to the series of FIGS. 1 and 6, a computer system and a method for estimating mental fatigue of a target individual according to an embodiment of the present invention will be described. Finally, referring to FIG. 7, a hardware configuration of a computer system according to one or more embodiments of the present invention will be described. In the following embodiments, mental fatigue is employed as a response variable for mental state estimation. However, in other embodiments, other mental states, such a mental workload, stress and sleepiness, may also be used as the response variable for the mental state estimation. In further embodiments, a mental state relating to mental health or some chronic medical condition, such as mental disorder, may also be used as the response variable for the mental state estimation in order to help medical diagnosis by professionals, such as doctors.

### Exemplary Embodiment

Now, referring to the series of FIGS. 1-5, a mental fatigue estimation system and methods for training a mental fatigue estimation model and estimating mental fatigue of a target individual according to an exemplary embodiment of the present invention is described.

FIG. 1 illustrates a block/flow diagram of a mental fatigue estimation system 100. As shown in FIG. 1, the mental fatigue estimation system 100 may include an eye tracking system 110, a raw training data store 120, a feature extractor 130, a training system 140, a model store 150, and an estimation engine 160.

The eye tracking system 110 may include an eye tracker 112 configured to acquire eye tracking data from a person P. The eye tracker 112 may be a device for measuring eye movement of the person P, which may be based on an optical tracking method using a camera or an optical sensor, electrooculogram (EOG) method, etc. The eye tracker 112 may be any one of non-wearable eye trackers and wearable eye trackers.

The person P may be referred to as a participant when the system 100 is in a training phase. The person P may be referred to as a target individual when the system 100 is in a test phase. The participant and the target individual may be same or may not be same, and may be any person in general. When a mental fatigue estimation model dedicated for a specific individual is requested, the participant for training may be identical to the specific individual who is also the target individual in the test phase.

The person P may watch a display screen S that shows a video and/or picture, while the eye tracker 112 acquires the eye tracking data from the person P. In a particular embodiment, the person P may be in natural-viewing conditions, where the person P watches freely a video and/or picture displayed on the display screen S while not performing any cognitive task. In an embodiment, unconstrained natural viewing of a video is employed as the natural-viewing situation. However, in other embodiments, any kind of natural viewing conditions, which may include unconstrained viewing of scenery through a window opened in a wall, vehicle, etc., can also be employed.

The raw training data store 120 may store one or more raw training data, each of which includes a pair of eye tracking data acquired from the person P and label information indicating mental fatigue of the person P at a period during which the eye tracking data is acquired. The label information may be given as subjective and/or objective measure, which may represent state of the mental fatigue (e.g., fatigue/non-fatigue) or degree of the mental fatigue (e.g., 0-10 rating scales).

The feature extractor 130 may read the eye tracking data from the raw training data store 120 in the training phase. The feature extractor 130 may receive the eye tracking data from the eye tracker 112 in the test phase. The feature extractor 130 may be configured to extract a plurality of eye movement features from the eye tracking data. In an embodiment, the plurality of eye movement features may include one or more base features and one or more extended features.

The base features can be extracted from the eye tracking data by using any known techniques. To extract the extended features, the feature extractor 130 may be configured to obtain information of eye movement of the person P in a predetermined coordinate system from the eye tracking data. The feature extractor 130 may be further configured to analyze the information of the eye movement to extract the one or more extended features of the eye movement defined in relation to a predetermined coordinate system.

In an embodiment, the information of the eye movement may be defined in a coordinate system that determines a point representing the eye movement by an angle and/or a distance with respect to a reference point. More detail about the base and extended features, extraction of the base and extended features and the coordinate system for the extended features will be described below.

In the training phase, the training system 140 may be configured to perform training of the mental fatigue estimation model using one or more training data. Each training data used by the training system 140 may include a pair of the plurality of eye movement features and the label information. The plurality of eye movement features may be extracted by the feature extractor 130 from the eye tracking data stored in the raw training data store 120. The label information may be stored in the raw training data store 120 in association with the eye tracking data that is used to extract the corresponding eye movement features.

The mental fatigue estimation model trained by the training system 140 may be a learning model that receives the plurality of eye movement features as input and performs classification or regression to determine a state or degree of the mental fatigue of the person P (e.g., the target individual).

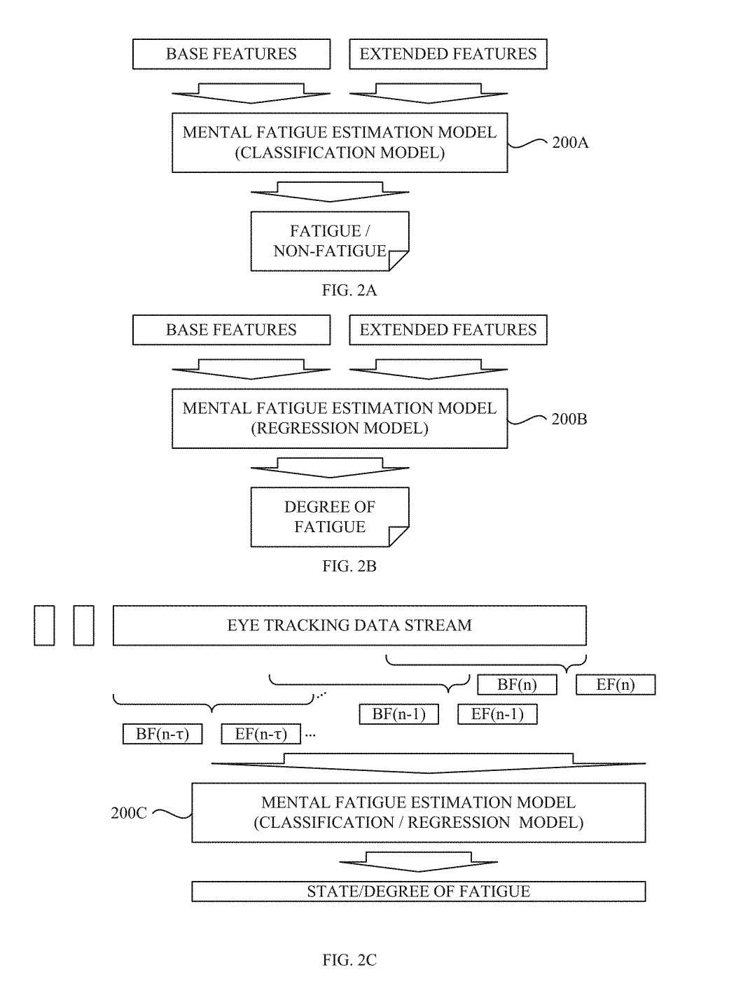

FIGS. 2A-2C depict examples of a mental fatigue estimation models 200A-200C according to one or more embodiments of the present invention. In a particular embodiment shown in FIG. 2A, the learning model may be a classification model 200A that receives the base and extended features as input and performs a classification task to determine a state of the mental fatigue as a discrete value (e.g., fatigue/non-fatigue). In another embodiment shown in FIG. 2B, the learning model may be a regression model 200B that receives the base and extended features as input and performs a regression task to determine a degree of the mental fatigue as a continuous value (e.g., 0-10 rating scales).

Any known learning models, such as ensembles of decision trees, SVM (Support Vector Machines), neural networks, etc., and corresponding appropriate machine learning algorithms can be employed.

Referring back to FIG. 1, the model store 150 may be configured to store the mental fatigue estimation model 200 trained by the training system 140. After training the mental fatigue estimation model 200, the training system 140 may save parameters of the mental fatigue estimation model 200 into the model store 150.

In the test phase, the estimation engine 160 may be configured to estimate the mental fatigue of the target individual P using the mental fatigue estimation model 200 stored in the model store 150. The estimation engine 160 may receive the base and extended features extracted from the eye tacking data of the target individual P and output the state or degree of the mental fatigue of the target individual P as an estimated result R.

In an embodiment using the classification model 200A, the estimation engine 160 may determine the state of the mental fatigue by inputting the base and extended features into the mental fatigue estimation model 200A. In another embodiment using the regression model 200B, the estimation engine 160 may determine the degree of the mental fatigue by inputting the base and extended features into the mental fatigue estimation model 200B. In an embodiment, the estimation engine 160 can perform mental fatigue estimation without knowledge relating to content of the video and/or picture displayed on the display screen S.

In an embodiment, it is assumed that the target individual P is watching the display screen S during acquisition of the eye tracking data, for simplicity. However, in other embodiments, the estimation engine 160 can switch a mode of the estimation from a task-performing mode using conventional mental fatigue estimation techniques to a natural viewing mode using the novel mental fatigue estimation and vice versa in response to being notified from an external system that is configured to detect situations of the target individual P.

In an embodiment, the training phase may be performed prior to the test phase. However, in another embodiment, the training phase and the test phase may be performed alternatively in order to improve estimation performance for a specific user. For example, the system 100 may inquire about user's tiredness (e.g., 0-10 rating scales) on a regular basis (e.g., just after start of work or study, and just before end of the work or study) to collect training data and update the mental fatigue estimation model by using newly collected training data.

In some embodiments, each of modules 120, 130, 140, 150 and 160 described in FIG. 1 may be implemented as, but not limited to, a software module including program instructions and/or data structures in conjunction with hardware components such as a processor, a memory, etc.; a hardware module including electronic circuitry; or a combination thereof. These modules 120, 130, 140, 150 and 160 described in FIG. 1 may be implemented on a single computer system, such as a personal computer, a server machine and a smartphone, or over a plurality of devices, such as a computer cluster of the computer systems in a distributed manner.

The eye tracking system 110 may be located locally or remotely to a computer system that implements the modules 120, 130, 140, 150 and 160 described in FIG. 1. The eye tracker 112 may be connected to the computer system via a computer-peripheral interface such as USB (Universal Serial Bus), Bluetooth™, etc. or through a wireless or wired network. Alternatively, the eye tracker 112 may be embedded in the computer system. In some embodiments, the eye tracking data may be provided to the computer system as a data file that is saved by a local or remote eye tracker, a data stream from a local eye tracker (connected to the computer system or embedded in the computer system), or a data stream via network socket from a remote eye tracker, which may be connected to or embedded in other remote computer systems, such as a laptop computer or smartphone. An existing camera included in the computer system may be utilized as a part of an eye tracker.

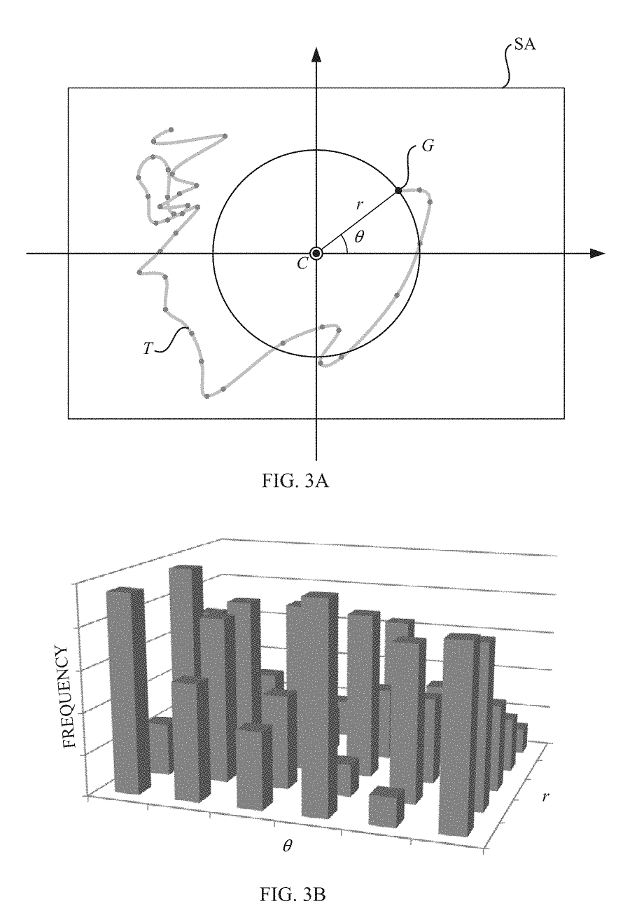

Hereinafter, referring to FIGS. 3A and 3B, the plurality of eye movement features used in the mental fatigue estimation system 100 will be described in more detail.

The eye tracking data acquired by the eye tracker 112 may include time series data of a point of gaze, information of blink and/or information of pupil. The time series data of the point of the gaze may include a component of fixation and a component of saccade. The fixation is the maintaining of the gaze on a location. The saccade is movement of the eyes between two or more phases of the fixation. The components of the fixation and the component of the saccade can be identified and separated by using any known algorithm including algorithms using velocity and/or acceleration thresholds, dispersion-based algorithms, area-based algorithms, etc.

The feature extractor 130 shown in FIG. 1 may be configured to extract eye movement features from the time series data of the point of the gaze, the information of the blink and/or the information of the pupil, as the base features. The feature extractor 130 may be further configured to extract other eye movement features from the time series data of the point of the gaze, as the extended features.

In an embodiment, the base features extracted from the saccade component and the extended features extracted from the fixation component can be employed. Such base features may include one or more eye movement features derived from at least one selected from a group including saccade amplitude, saccade duration, saccade rate, inter-saccade interval (mean, standard deviation and coefficient), mean velocity of saccade, peak velocity of saccade, to name but a few.

However, the base features may not be limited to the aforementioned saccade features. In other embodiments, other features derived from at least one of blink duration, blink rate, inter-blink interval (mean, standard deviation and coefficient), pupil diameter, constriction velocity, constriction amplitude of pupil, etc. may be used as the base feature in place of or in addition to the aforementioned saccade features.

FIG. 3A describes an example of a coordinate system used for extracting one or more extended features. FIG. 3B depicts an example of the one or more extended features defined in relation to the coordinate system shown in FIG. 3A.

The examples of the extended features described in FIGS. 3A and 3B are based on the time series data of the point of the gaze. The time series data for the extended features may include at least fixation component. In an embodiment, the time series data for the extended features may be the fixation component separated from whole time series data of the point of the gaze if possible. In another embodiment, the time series data of the point of the gaze may be treated as the fixation component if separation of the fixation component is not conducted.

Typically, the point of the gaze acquired by the eye tracker 112 may be defined in a Cartesian coordinate system on the display screen S when the eye tracker 112 is a non-wearable eye tracker. To extract the extended features, the feature extractor 130 first obtains the time series data of the point of the gaze in a polar coordinate system by performing coordinate transformation from the original coordinate system to the polar coordinate system.

The polar coordinate system may determine the point of the gaze G by an angle θ and a distance r with respect to a reference point C. The reference point C may be related to a center of an area SA corresponding to an object showing a scene, which may have a planar or curved surface facing toward the person P. In the describing embodiment, the object that is seen by the person P and defines the reference point C may be the display screen S showing a video and/or picture as the scene and the reference point C may be placed at the center of the display screen S.

When the eye tracker 112 is the non-wearable eye tracker, calibration of the reference point C may be conducted in advance. When the eye tracker 112 is not fixed to the display screen S (e.g., a desktop eye tracker), positional relationship (e.g., relative position, relative angle) between the display screen S and the eye tracker 112 may be given for each installation condition prior to the calibration of the reference point C. The calibration of the reference point C can be done by directing the person P to look at a specific point such as the center of the display screen S during a calibration phase, for example.

Also when the eye tracker 112 is the wearable (e.g., a head mounted eye tracker), the point of the gaze acquired by the eye tracker 112 may be defined in a coordinate system on a camera which may be fixed to the head of the person P. In this case, the display screen S and its center may be detected in an image obtained from the camera and the coordinate system for the point of the gaze may be converted into the coordinate on the display screen S prior to the coordinate transformation to the polar cordinate system.

However, the object defining the reference point may not be limited to the center of the aforementioned display screen S. In another embodiment with the unconstrained viewing of the scenery through the window, the object defining the reference point may be the window through which the person P can view the scenery as the scene, for example.

In the polar coordinate system shown in FIG. 3A, the time series data of the point of the gaze T with a certain time length may draw a trajectory. The feature extractor 130 may analyze the time series data of the point of the gaze T defined in the polar coordinate system to extract a frequency distribution of fixation (r, θ) as the one or more extended features. As shown in FIG. 3B, the frequency distribution of the fixation (r, θ) may include a plurality of cells or meshes, each of which holds a (relative) frequency of the fixation detected at a region designated by the row and the column from the time series data of the point of the gaze T.

However, in other embodiments, the frequency distribution of the fixation (r) and the frequency distribution of the fixation (θ) calculated independently from the time series data of the point of the gaze T may be used as the extended features in place of or in addition to the frequency distribution of the fixation (r, θ) in 2D form. Also entropy and/or static (e.g., mean, median, standard deviation, etc.) of the fixation (r, θ) may also be used as the extended features in addition to the frequency distribution.

The frequency distribution of the fixation (r, θ) may be used as a part of or whole of explanatory variables of the mental fatigue estimation model 200. Conventionally, features that originated from the gaze during a task has not been used for mental fatigue estimation since the person tends to follow targets during a task, such as a driving task, which may include forward vehicles, obstacles and pedestrians for the driving task. Thus, the frequency distribution of the fixation (r, θ) may be suitable for natural-viewing conditions.

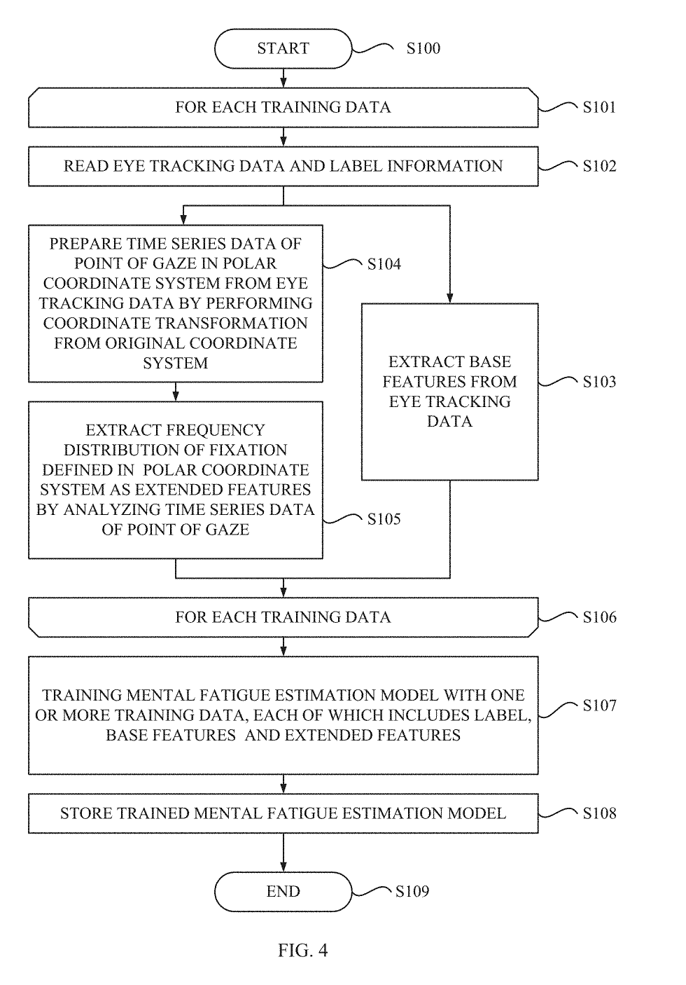

Hereinafter, referring to FIG. 4, a novel process for learning the mental fatigue estimation model 200 will be described.

FIG. 4 shows a flowchart depicting a process for learning the mental fatigue estimation model 200 in the mental fatigue estimation system 100 shown in FIG. 1. Note that the process shown in FIG. 4 may be performed by a processing unit that implements the feature extractor 130 and the training system 140 shown in FIG. 1.

Also note that the saccade features extracted from the saccade component is employed as the base features and the frequency distribution of the fixation extracted from the fixation component is employed as the extended features in the process shown in FIG. 4. However, the base features may not be limited to the saccade features; other features, such as blink features and/or pupil features, may also be used as the base feature in place of or in addition to the saccade features. The extended features may not be limited to merely the frequency distribution of the fixation; entropy and/or statics (e.g., mean, median, standard deviation, etc.) of the fixation may also be used as the extended features in addition to the frequency.

The process shown in FIG. 4 may begin at step S100 in response to receiving a request for training with one or more arguments. One of the arguments may specify a group of the raw training data to be used for training. The processing from step S101 to S106 may be performed for each training data to be prepared.

At step S102, the processing unit may read the eye tracking data and corresponding label information from the raw training data store 120 and set the label information into the training data. At step S103, the processing unit may extract the saccade features from the saccade component in the eye tracking data. The extracted saccade features may be set into the training data as the based features.

At step S104, the processing unit may prepare the time series data of the point of the gaze in the polar coordinate system from the eye tracking data by performing the coordinate transformation from the original Cartesian coordinate. At step S105, the processing unit may extract the frequency distribution of the fixation defined in the polar coordinate system by analyzing the time series data of the point of the gaze in the eye tracking data. During the course of the analysis, the number of the occurrences of the fixation may be counted for each class defined by ranges of the angle θ and/or the distance r. The extracted frequency distribution of the fixation may be set into the training data as the extended features.

During the loop from the step S101 to the step S106, the processing unit may prepare one or more training data by using the given raw training data. If the processing unit determines that a desired amount of the training data has been prepared or analysis of all given raw training data has been finished, the process may exit the loop and the process may proceed to step S107.

At step S107, the processing unit may perform training of the mental fatigure estimation model 200 by using appropriate machine laming algorithm with the prepared training data. Each training data may include the label information obtained at step S102, the base features (e.g., the saccade features) obtained at the step S103 and the extended features (e.g., the frequency distribution of the fixation) obtained at the step S105. In a particular embodiment using an ensamble of decision trees as the learning model, the random forest algoritm can be applied.

At step S108, the processing unit may store the trained parameter of the mental fatigure estimation model into the model store 150 and the process may end at step S109.

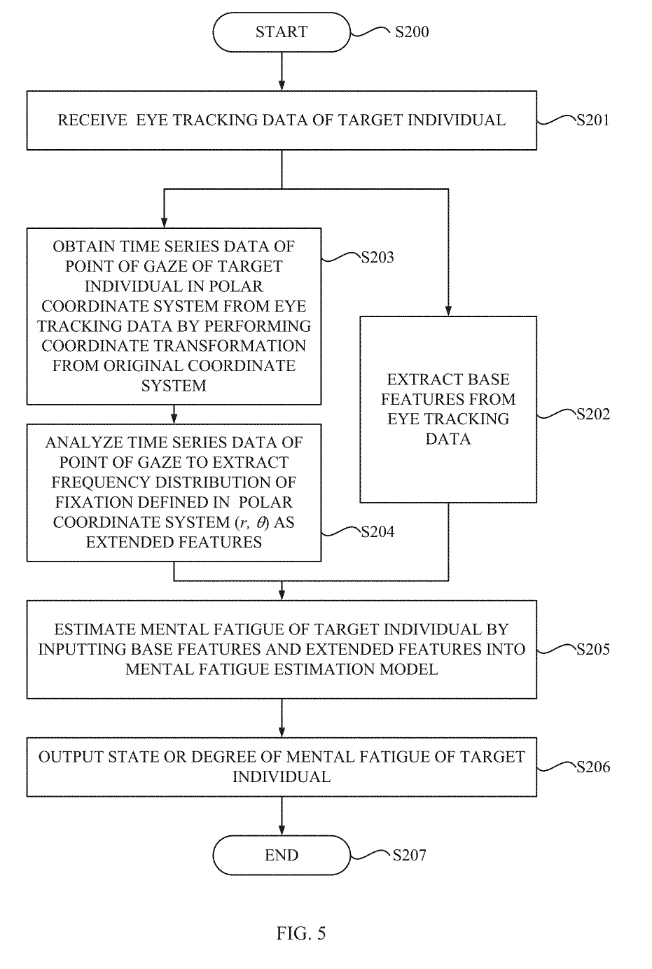

Hereinafter, referring to FIG. 5, a novel process for estimating the mental fatigue using the mental fatigue estimation model 200 trained by the process shown in FIG. 4 will be described.

FIG. 5 shows a flowchart depicting a process for estimating the mental fatigue in the mental fatigue estimation system 100 shown in FIG. 1. Note that the process shown in FIG. 5 may be performed by a processing unit that implements the feature extractor 130 and the estimation engine 160 shown in FIG. 1. Also note that the base and extended features used in the process shown in FIG. 5 may be identical to those used in the process shown in FIG. 4.

The process shown in FIG. 5 may begin at step S200 in response to receiving a request for estimating mental fatigue of a target individual P. At step S201, the processing unit may receive eye tracking data acquired by the eye tracker 112 from the target individual P. The eye tracking data may have a certain time length. At step S202, the processing unit may extract the saccade features from the saccade component in the eye tracking data, as the based feature.

At step S203, the processing unit may obtain time series data of the point of the gaze of the target individual P in the polar coordinate system from the eye tracking data by performing the coordinate transformation from the original Cartesian coordinate. At step S204, the processing unit may analyze the time series data of the gaze in the eye tracking data to extract the frequency distribution of the fixation defined in the polar coordinate system as extended features.

At step S205, the processing unit may estimate mental fatigue of the target individual P by inputting the base features (e.g., the saccade features) and the extended features (e.g., the frequency distribution of the fixation) into the mental fatigue estimation model 200. At step S206, the processing unit may output the state or degree of the mental fatigue of the target individual P and the process may end at step S207.

In a particular embodiment using an ensamble of trees as the classification model, the state of the mental fatigue may be determined by taking majority vote of the trees in the ensamble. In another embodiment using an ensamble of trees as the regression model, the degree of the mental fatigue may be determined by averaging the predictions from all the trees in the ensamble.

In the aforementioned embodiment, the base features and the extended features may be calculated from whole time seris data of the given eye tracking data. However, ways of calculating the base features and the extended features may not be limited to the aforementioned embodiments. In another embodiment, the feature extractor 130 may receive from the eye tracker 112 a part of eye tracking stream data within a certain time window and extract a frame of the base and extended features from the received part of the eye tracking stream data. Then, the estimation engine 160 may continuously output each frame holding an estimated result in response to receiving each frame of the base and extended features.

FIG. 2C depicts an example of the mental fatigue estimation model 200C used in an embodiment. The mental fatigue estimation model 200C shown in FIG. 2C may receive a series of feature frames, each of which includes the base feature BF(i) and extended features EF(i) calculated from each corresponding part of the eye tracking stream data within a predetermined time window. The estimation engine 160 may continuously output each result frame for current timing (n) in response to receiving the series of the feature frames (n-τ, . . . , n-1, n), which may include BF(n-τ), EF(n-τ), . . . , BF(n-1), EF(n-1), BF(n), and EF(n) as shown in FIG. 2C.

### Alternative Embodiment

In the aforementioned exemplary embodiment, the mental fatigue estimation system 100 estimates the mental fatigue of the target individual P by using the trained mental fatigue estimation model 200. Now, referring to the series of FIGS. 1 and 6, a computer system and method for estimating mental fatigue of a target individual P according to an alternative embodiment of the present invention will be described in which a mental fatigue estimation system 100 estimates the mental fatigue of the target individual using a predetermined rule.

A block/flow diagram of a mental fatigue estimation system 100 according to the alternative embodiment is similar to that of the exemplary embodiment shown in FIG. 1. Since the configuration of the alternative embodiment has similarity to the exemplary embodiment, hereinafter, mainly features different from the exemplary embodiment will be described.

Further referring to FIG. 1, the block diagram of the mental fatigue estimation system 100 according to the alternative embodiment is illustrated in a dashed rectangular. As shown in FIG. 1, the mental fatigue estimation system 100 according to the alternative embodiment may include an eye tracking system 110, a feature extractor 130, and an estimation engine 160.

The feature extractor 130 according to the alternative embodiment may be configured to extract features of eye movement from the eye tracking data received from the eye tracker 112. In a particular embodiment, the frequency distribution of the fixation in the polar coordinate system may be employed as the features of the eye movement.

The estimation engine 160 according to the alternative embodiment may be configured to estimate the mental fatigue of the target individual P using the predetermined rule. The estimation engine 160 may receive the feature of the eye movement from the feature extractor 130 and output a state of the mental fatigue of the target individual P as an estimated result R.

In a particular embodiment, the estimation engine 160 may determine whether or not the frequency distribution of the fixation indicates a bias towards a specific area in the coordinate system using the predetermined rule. The predetermined rule may describe a condition for detecting a bias toward the reference point in the polar coordinate system (e.g., r tends to be zero) and/or a bias toward a horizontal axis in the coordinate system (e.g., θ tends to be 0 or 180 degrees). Such rule may be obtained from eye tracking experiments in the natural viewing condition.

In the alternative embodiment, the frequency distribution of the fixation may include a plurality of elements, each of which holds a frequency of the fixation detected at a respective region divided from the polar coordinate system. For example, if the polar coordinate system is divided into several regions including simply a central region, a horizontal region and a peripheral region by using the angle θ and the distance r, for each of which frequency is counted, the condition for detecting the bias can be simply designed by using one or more empirical threshold values to the frequency distribution of the fixation.

FIG. 6 shows a flowchart depicting a process for estimating the mental fatigue of the target individual P according to the alternative embodiment. Note that the process shown in FIG. 6 may be performed by a processing unit that implements the feature extractor 130 and the estimation engine 160 in the rectangular shown in FIG. 1. Also note that the process shown in FIG. 6 may use the frequency distribution of the fixation extracted from the fixation component of the eye tracking data as the features of the eye movement.

The process shown in FIG. 6 may begin at step S300 in response to receiving a request for estimating the mental fatigue of the target individual P. At step S301, the processing unit may receive eye tracking data acquired from the target individual P.

At step S302, the processing unit may obtain the time series data of the point of the gaze of the target individual P in the polar coordinate system from the eye tracking data. At step S303, the processing unit may analyze the time series data of the point of the gaze to extract the frequency distribution of the fixation defined in the polar coordinate system as the feature of the eye movement.

At step S304, the processing unit may determine whether or not the frequency distribution indicates a bias toward center and/or bias toward the horizontal axis on the basis of the predetermined rule in order to estimate the mental fatigue of the target individual. The estimation engine 160 may determine that the state of the mental fatigue is “fatigue” state when the frequency distribution indicates the bias toward the reference point or the horizontal axis.

At step S305, the processing unit may output the state of the mental fatigue of the target individual P and the process may end at step S306.

### Experimental Studies

A program implementing the system shown in FIG. 1 and the process shown in FIGS. 4 and 5 according to the exemplary embodiment was coded and executed for given training samples and test samples.

The samples were obtained from a total of 15 participants (7 females, 8 males; 24-76 years; mean (SD) age 51.7 (19.9) years). The eye tracking data was acquired from each participant while the participant was watching a video clip of 5 minutes before and after doing a mental calculation task of approximately 35 minutes by hearing questions, which required no visual processing. Each 5-min phase for video watching consisted of nine short video clips of 30 seconds. The eye tracking data of each 30 seconds obtained between breaks was used as one sample. The states of the mental fatigue of the participants were confirmed by observing statistically significant increment in both of subjective measure (0-10 rating scales) and objective measure (pupil diameter). The eye tracking data collected before the mental calculation task was labelled as “non-fatigue” and the eye tracking data collected after the task was labelled as “fatigue”. Thus, the numbers of the samples for both “non-fatigue” and “fatigue” states were 9*15=135, respectively.

Twenty-one features derived from saccade amplitude, saccade duration, saccade rate, inter-saccade interval (mean, standard deviation, and coefficient of variance), mean saccade velocity (mean and median), blink duration, blink rate, blink duration per minute, inter-blink interval (mean, standard deviation, and coefficient of variance), a diameter of a pupil of each eye, constriction velocity of the pupil of each eye, and constriction amplitude of the pupil of each eye were employed as the base features. The frequency distribution of the fixation having (36 ranges of the angle θ, 8 ranges of the distance r) was employed as the extended features.

A classification model of support vector machine (SVM) with a radial basis function kernel and an improved SVM-recursive feature elimination algorithm with a correlation bias reduction strategy in the feature elimination procedure was used as the mental fatigue estimation model.

As an example, the classification model was trained by using both of the base and extended features of the prepared training samples. As a comparative example, the classification model was trained by using merely the base features of the prepared training samples. Unless otherwise noted, any portions of the classification model except for the input were approximately identical between the example and the comparative example.

Classification performance of the mental fatigue estimation using the classification model was evaluated by 2-class classification accuracy, which was calculated from test samples according to 10-fold cross-validation method.

The evaluated results of the example and the comparative example are summarized as follows:

Classification accuracy (chance 50%)

| Comparative Example | Example | |
|---|---|---|
| (w/o extended features) | (w/ extended features) | improvement |
| 0.77 | 0.83 | approximately 6% |

By comparison with the result of the comparative example, the accuracy of the example increased by approximately 6%.

Computer Hardware Component

Referring now to FIG. 7, a schematic of an example of a computer system 10, which can be used for the mental fatigue estimation system 100, is shown. The computer system 10 shown in FIG. 7 is implemented as a computer system. The computer system 10 is only one example of a suitable processing device and is not intended to suggest any limitation as to the scope of use or functionality of embodiments of the invention described herein. Regardless, the computer system 10 is capable of being implemented and/or performing any of the functionality set forth hereinabove.

The computer system 10 is operational with numerous other general purpose or special purpose computing system environments or configurations. Examples of well-known computing systems, environments, and/or configurations that may be suitable for use with the computer system 10 include, but are not limited to, personal computer systems, server computer systems, thin clients, thick clients, hand-held or laptop devices, in-vehicle devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system 10 may be described in the general context of computer system-executable instructions, such as program modules, being executed by a computer system. Generally, program modules may include routines, programs, objects, components, logic, data structures, and so on that perform particular tasks or implement particular abstract data types.

As shown in FIG. 7, the computer system 10 is shown in the form of a general-purpose computing device. The components of the computer system 10 may include, but are not limited to, a processor (or processing unit) 12 and a memory 16 coupled to the processor 12 by a bus including a memory bus or memory controller, and a processor or local bus using any of a variety of bus architectures.

The computer system 10 typically includes a variety of computer system readable media. Such media may be any available media that is accessible by the computer system 10, and it includes both volatile and non-volatile media, removable and non-removable media.

The memory 16 can include computer system readable media in the form of volatile memory, such as random access memory (RAM). The computer system 10 may further include other removable/non-removable, volatile/non-volatile computer system storage media. By way of example only, the storage system 18 can be provided for reading from and writing to a non-removable, non-volatile magnetic media. As will be further depicted and described below, the storage system 18 may include at least one program product having a set (e.g., at least one) of program modules that are configured to carry out the functions of embodiments of the invention.

Program/utility, having a set (at least one) of program modules, may be stored in the storage system 18 by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments of the invention as described herein.

The computer system 10 may also communicate with one or more peripherals 24, such as a keyboard, a pointing device, a car navigation system, an audio system, etc.; a display 26; one or more devices that enable a user to interact with the computer system 10; and/or any devices (e.g., network card, modem, etc.) that enable the computer system 10 to communicate with one or more other computing devices. Such communication can occur via Input/Output (I/O) interfaces 22. Still yet, the computer system 10 can communicate with one or more networks such as a local area network (LAN), a general wide area network (WAN), and/or a public network (e.g., the Internet) via the network adapter 20. As depicted, the network adapter 20 communicates with the other components of the computer system 10 via bus. It should be understood that, although not shown, other hardware and/or software components could be used in conjunction with the computer system 10. Examples, include, but are not limited to: microcode, device drivers, redundant processing units, external disk drive arrays, RAID systems, tape drives, and data archival storage systems, etc.

Computer Program Implementation

The present invention may be a computer system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more aspects of the present invention has been presented for purposes of illustration and description, but is not intended to be exhaustive or limited to the invention in the form disclosed.

Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for estimating a mental state of a target individual, the method comprising:
obtaining information of eye movement of the target individual in a coordinate system, the coordinate system determining a point representing the eye movement by an angle and/or a distance with respect to a reference point related to a center of an object showing a scene;
analyzing the information of the eye movement to extract a feature of the eye movement defined in relation to the coordinate system; and
estimating the mental state of the target individual using the feature of the eye movement.

2. The method of claim 1, wherein the mental state is mental fatigue, the information of the eye movement is time series data of a point of gaze obtained from the target individual, the coordinate system determines a position of the point of the gaze by the angle and the distance, and the feature of the eye movement includes a frequency distribution of the point of the gaze.

3. The method of claim 2, wherein estimating comprises determining a state or a degree of the mental fatigue by using a learning model, the learning model receiving the frequency distribution as input and performing classification or regression.

4. The method of claim 3, wherein the learning model receives one or more eye movement features selected from a group including saccade amplitude, saccade duration, saccade rate, inter-saccade interval, mean velocity of saccade, peak velocity of saccade, blink duration, blink rate, inter-blink interval, pupil diameter, constriction velocity and constriction amplitude of a pupil, in addition to the frequency distribution.

5. The method of claim 3, wherein the learning model is trained using one or more training data, each training data including label information indicating the mental fatigue of a participant and the frequency distribution extracted from the time series data of the point of gaze obtained from the participant.

6. The method of claim 2, wherein estimating comprises determining whether or not the frequency distribution indicates a bias toward the reference point and/or a bias toward a horizontal axis in the coordinate system to estimate the mental fatigue.

7. The method of claim 1, wherein the information of the eye movement is time series data of a point of gaze including a component of fixation, or is time series data of a component of fixation separated from a component of saccade.

8. The method of claim 1, wherein the information of the eye movement is acquired by an eye tracking device from the target individual in a natural-viewing condition.

9. The method of claim 8, wherein the object is a screen showing a video and/or a picture and the natural-viewing condition is a natural viewing condition of the video and/or the picture, the estimating being performed without knowledge relating to content of the video and/or the picture.

10. A computer-implemented method for training a learning model used for estimating a mental state of a target individual, the method comprising:
preparing information of eye movement of a participant in a coordinate system, the coordinate system determining a point representing the eye movement by an angle and/or a distance with respect to a reference point related to a center of an object showing a scene;
extracting a feature of the eye movement defined in relation to the coordinate system by analyzing the information of the eye movement; and
training the learning model using one or more training data each including the feature of the eye movement and corresponding label information indicating the mental state of the participant.

11. The method of claim 10, wherein the mental state is mental fatigue, the information of the eye movement is time series data of a point of gaze obtained from the participant, the coordinate system determines a position of the point of the gaze by the angle and the distance, and the feature of the eye movement includes a frequency distribution of the point of the gaze.

12. The method of claim 11, wherein the learning model receives the frequency distribution of the target individual as input and performs classification or regression to determine a state or a degree of the mental fatigue of the target individual.

13. The method of claim 11, wherein each training data includes one or more eye movement features selected from a group including saccade amplitude, saccade duration, saccade rate, inter-saccade interval, mean velocity of saccade, peak velocity of saccade, blink duration, blink rate, inter-blink interval, pupil diameter, constriction velocity and constriction amplitude of a pupil, in addition to the frequency distribution.

14. A computer system for estimating a mental state of a target individual, by executing program instructions, the computer system comprising:
a memory tangibly storing the program instructions; and
a processor in communications with the memory, wherein the processor is configured to:
obtain information of eye movement of the target individual in a coordinate system, the coordinate system determining a point representing the eye movement by an angle and/or a distance with respect to a reference point related to a center of an object showing a scene;
analyze the information of the eye movement to extract a feature of the eye movement defined in relation to the coordinate system; and
estimate the mental state of the target individual using the feature of the eye movement.

15. The computer system of claim 14, wherein the mental state is mental fatigue, the information of the eye movement is time series data of a point of gaze obtained from the target individual, the coordinate system determines a position of the point of the gaze by the angle and the distance, and the feature of the eye movement includes a frequency distribution of the point of the gaze.

16. The computer system of claim 15, wherein the processor is further configured to use a learning model to determine a state or a degree of the mental fatigue, the learning model receiving the frequency distribution as input and performing classification or regression.

17. The computer system of claim 16, wherein the learning model is trained using one or more training data, each training data including label information indicating mental fatigue of a participant and the frequency distribution extracted from the time series data of the point of gaze obtained from the participant.

18. The computer system of claim 15, wherein the processor is further configured to determine whether or not the frequency distribution indicates a bias toward the reference point and/or a bias toward a horizontal axis in the coordinate system to estimate the mental fatigue.

19. The computer system of claim 14, wherein the information of the eye movement is acquired by an eye tracking device from the target individual in a natural viewing condition.

20. A computer program product for estimating a mental state of a target individual, the computer program product comprising a non-transitory computer readable storage medium having program instructions embodied therewith, the program instructions executable by a computer to cause the computer to perform the method of claim 1.
